(19) United States
(12) Patent Application Publication
(10) Pub. No.: US 2011/0239332 A1
(43) Pub. Date: Sep. 29, 2011

(54) PROPAGATION OF SWITCHGRASS AND MISCANTHUS

(76) Inventor: Ronald E. Rothrock, JR., West Edmeston, NY (US)

(21) Appl. No.: 13/055,634

(22) PCT Filed: Jul. 22, 2009
(86) PCT No.: PCT/US09/51355

§ 371 Date: Jun. 14, 2011

Related U.S. Application Data
(60) Provisional application No. 61/082,951, filed on Jul. 23, 2008.

(51) Int. Cl. A01H 5/00; A01G 31/00; A01C 14/00

(52) U.S. Cl. 800/298; 47/59 S; 111/100

(57) ABSTRACT

Methods and materials for clonally increasing switchgrass and Miscanthus shoot multiplication rate in culture media are disclosed. Clonal multiplication may be carried out at rates acceptable for various commercial applications.

### CROSS-REFERENCE TO RELATED APPLICATIONS

This application claims the benefit of U.S. Provisional Application Ser. No. 61/082,951, filed on Jul. 23, 2008. The disclosure of the prior application is incorporated by reference in its entirety.

### TECHNICAL FIELD

The present invention relates to methods of clonal propagation of energy crop plants in culture media. In particular, the present invention relates to methods for culturing stem nodes and increasing the shoot multiplication rate of Panicum virgatum L. (switchgrass) and Miscanthus × giganteus (Miscanthus) in tissue culture.

### BACKGROUND

Propagation of plants by tissue culture methods has been successfully applied to many commercial crop plants. In general, tissues or cells of a desired plant are grown on a medium which causes multiplication of the tissues. These multiplied tissues are then divided and grown on other media which cause rapid multiplication of tissue such as shoots, which can be regenerated in to whole plants. The efficiency of the rapid multiplication step is important to achieving levels of multiplication of plants that are economically viable. The present invention is based in part on the discovery of processes that can achieve commercially acceptable rates of shoot multiplication in culture media thus accelerating the rate at which whole plants can be regenerated.

Limitations on shoot multiplication rates are a significant problem encountered in the in vitro culture of plant tissues, particularly when plants are propagated via tissue culture on a commercial scale. The shoot multiplication rate of cultured plant material is often limited when the culture media does not have the optimal combination of components or contains harmful amounts of deleterious substances such as phenolic substances, tannins or other hydroxyphenols. The result of this is that the multiplication of plant tissue in culture is inhibited, frequently irreparably. Thus, increasing multiplication rates represents a serious obstacle to the propagation of plants and/or plant tissues in culture on a commercially viable scale. Multiplication rates insufficient for viable commercialization have been observed in a wide range of plant tissues and plant cell cultures and in particular in grass species used for energy crops, which are expected to require rapid and high rates of multiplication to meet demands for production. Methods for multiplication of plants in culture media can be useful to meet the demand for rapid agricultural production of uniform and superior energy crops and/or energy crops that are highly suitable for regions having differences in factors such as climate, soil, temperature, rainfall or prevalence of diseases and pests.

One plant known to suffer from limited shoot multiplication rates in culture media is the commercially important energy crop plant, switchgrass. Switchgrass is a perennial grass with tall unbranched stems or canes.

A number of approaches have been taken in attempting to address the problem of multiplication rates. These include varying the combinations of hormones in shoot multiplication media and multiplying shoots in solid or liquid media. However, no single approach has proved satisfactory to multiply switchgrass shoots in culture media at a rate that fits commercial production demands.

Miscanthus is another plant for which propagation has turned out to be challenging. At northern latitudes Miscanthus cultivars often do not produce seeds, and propagation has traditionally been accomplished by rhizome planting. Digging out rhizomes for planting is more labor intensive and expensive and the rhizome materials are often sufficient only for few acres of planting. Methods of propagating Miscanthus using materials for planting either in the field or in the greenhouse are needed for large scale commercial cultivation of the species.

### SUMMARY

The invention features methods and materials related to switchgrass and Miscanthus establishment, multiplication, and rooting in culture media. The methods described herein can be used to increase the multiplication rate in vitro of shoots made by such methods.

In one aspect, the invention features a method of multiplying switchgrass shoots. The method comprises growing a plurality of switchgrass shoots at about 31° C. in a multiplication media comprising Cu⁺⁺ at a concentration of from about 0.040 μM to about 45 μM. The Cu⁺⁺ concentration can be 0.040, 0.1, 0.20, 0.40, 1, 3, 5, 10, 15, 20, 25, 30, 35, 40, or 45 μM. The Cu⁺⁺ can be added to the media as CuSO₄.7H₂O. The switchgrass shoots can multiply during the growing step at a rate of at least ten-fold in three weeks. The switchgrass shoot multiplication rate can be continuous. The multiplication media can further comprise 6 g/L agar. The multiplication media can further comprise MS plus vitamins, 3% sucrose, hormones BAP, TDZ, NAA, and PBZ in a concentration ratio of 20:1:4:4, under conditions of pH 5.7, 16 hours of light per day, and 35-75 μE of light.

In some embodiments, the plurality of shoots are formed by differentiation from a stem section containing at least a portion of a node in an establishment media, prior to transfer to multiplication media. The establishment media can comprise MS plus vitamins, 3% sucrose, 1 mg/L CuSO₄.5H₂O, 5 mg/L BAP, 0.25 mg/L TDZ and, in some case, 1.0 mg/L NAA and 1.0 mg/L PBZ. The establishment media can further comprise 6 g/L agar. The plurality of shoots can be grown under conditions of pH 5.7, 16 hours of light per day, and 50-100 μE of light. The shoots can be removed from the establishment media after two weeks and separated from the node. In some embodiments, the shoots are removed from the establishment media at 3 cm long and separated from the node.

The shoots can be placed in a rooting media after the multiplication step. The rooting media can comprise MS plus vitamins, 3% sucrose, 1 mg/L CuSO₄.5H₂O, and 1.0 mg/L NAA, and the plants can be grown on rooting media under conditions of pH 5.7, 16 hours of light per day, and 50-100 μE of light. The rooting media can further comprise 6 g/L agar. The method can further comprise the step of regenerating a plurality of plants from the shoots placed on the rooting media, in which shoots placed on the rooting media are multiplied in the multiplication step from a single stem section containing at least a portion of a node. The invention also features a plant or progeny thereof made by such a method.

In another aspect, the invention features a method of making hybrid switchgrass seeds. The method comprises planting a first clonal population of switchgrass plants and a second clonal population of switchgrass plants in pollinating proximity to each other. Both the first and the second clonal populations of plants are regenerated from switchgrass shoots according to the methods described herein. Seeds produced on plants of the first clonal population, the second clonal population, or both the first and second clonal populations are collected, thereby making hybrid switchgrass seeds. The switchgrass plants of the first clonal population can be male sterile plants and seed can be collected from plants of the first clonal population. The switchgrass plants of the first clonal population can be transgenic plants.

In another aspect, the invention features a method for clonally propagating Miscanthus under conditions of 50-100 μE of light, 16 hours of light per day and 31° C. The method comprises growing stem sections that include at least a portion of a node in an semi-solid establishment media at pH 5.6 with 6 g/L agar, or a liquid establishment media at pH 3.3, to form shoots. The establishment media can comprise MS plus vitamins, 30 g/L sucrose (87.7 mM), 3.7 micro-Molar MgCl₂ (750 mg/L of MgCl₂.6H₂O), 5 mg/L BAP (22.2 micro-Molar), and 0.25 mg/L NAA (1.3 micro-Molar). The shoots that form are then cultured in a semi-solid multiplication media at pH 5.7 with 6 g/L agar, or a liquid multiplication media at pH 3.0, to multiply the shoots. The multiplication media can comprise MS plus vitamins, 3% sucrose, 5.0 mg/L BAP, 0.25 mg/L TDZ, and 1.0 mg/L PBZ. The shoots can multiply at a rate of at least eight-fold every three weeks, or at a rate that is continuous, i.e., can be maintained for a desired extended period of time such as 1 month, 2 months, 3 months, 6 months, 1 year, 2 years, 5 years or 10 years. After culturing on multiplication media, the resulting multiplied shoots can be grown in a rooting media comprising MS plus vitamins, 3% sucrose, and 10.0 mg/L NAA with 6 g/L agar at pH 5.7 to form Miscanthus plantlets. The stem sections can be about 3 cm long. The stem sections can be cut in cross-section and longitudinally. The invention also features a plant or progeny thereof made by such a method.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description.

### DETAILED DESCRIPTION

### I. Definitions:

As used herein, the term ‘node’ refers to a joint or notch on the stem or culm at which point a leaf can be attached and above which a single nodal bud is present.

As used herein, the term ‘CuSO₄.5H₂O’ refers to the amount of solid copper sulfate pentahydrate that is added when preparing a media.

### II. Propagation of Switchgrass In Vitro

Switchgrass propagation methods described herein generally comprise three main stages: growing cuttings in/on an establishment media, growing shoots in/on a multiplication media, and rooting shoots in/on a rooting media. In some embodiments, the methods do not include a callus phase.

The switchgrass stem is roughly circular or oval in cross-section, and comprises a series of joints each of which in turn comprises a node and an internode. Each node comprises a lateral bud in an axial leaf, a band containing root primordia, and a growth ring. In the methods of clonal mass propagation of switchgrass shoots described herein the starting material is stem segments having at least a portion of a node. The starting material is typically harvested from mature healthy plants and segments of stem are cut (in cross section) that comprise at least one node. These segments are then typically cut longitudinally. Care is taken to sterilize the stems, equipment and growing containers used in the tissue culture methods. These segments are then typically cut longitudinally. The media described herein and components thereof typically can be substituted with other media and components that are known in the art. For example, N6 media can be used instead of MS. A clear and sterile container is suitable for propagation.

### A. Establishment Media

In the methods described herein, switchgrass shoots typically are differentiated from a stem section containing at least a portion of a node in an establishment media. In some preferred embodiments, establishment media comprises MS plus vitamins, 3% sucrose, 1 mg/L CuSO₄.5H₂O, 5 mg/L BAP (Benzylaminopurine) and 0.25 mg/L TDZ (Thidiazuron) under conditions of pH 5.7, 31° C., 16 hours of light per day, and 50-100 μE of light. In embodiments where the media is a semi-solid, agar can be used. The agar can be added in about 3, 4, 5, 6, 7, 8, or 9 g/L. In some embodiments, the amount and/or concentrations of hormones may be different as described herein. In some embodiments, the ratio of hormones remains constant (i.e., 1 mg/L CuSO₄.5H₂O, 5 mg/L BAP and 0.25 mg/L TDZ). In some embodiments, the establishment media, and other media described herein, can comprise MS, MS plus vitamins, or other media known in the art such as Gamborgs B5 vitamins. In some embodiments, establishment media comprises about 0.5%, 1%, 2%, 3%, 4%, 5%, 6%, 7%, 8%, 9%, or 10% sucrose. In some embodiments, establishment media comprises about 0.01, 0.05, 0.1, 0.5, 1, 2, 3, 4, 5, 6, 7, 8, 9, or 10 mg/L of CuSO₄.5H₂O. In some embodiments, establishment media comprises Cu+ or Cu++ at a μM concentration of about 0.040, 0.100, 0.200, 0.400, 1, 3, 5, 10, 15, 20, 25, 30, 35, 40, or 45 μM or ranges therebetween. In some embodiments, establishment media comprises about 1, 2, 3, 4, 5, 6, 7, 8, 9, or 10 mg/L BAP. In some embodiments, BAP can be replaced all or in part with kinetin at about 1, 2, 3, 4, 5, 6, 7, 8, 9, or 10 mg/L. In some embodiments, BAP can be replaced all or in part with zeatin at about 1, 2, 3, 4, 5, 6, 7, 8, 9, or 10 mg/L. In some embodiments, establishment media comprises about 0.01, 0.1, 0.25, 0.75, 1, 1.5, 2, 2.5, 3, 3.5, 4, or 4.5 mg/L TDZ. In some embodiments, establishment media has a pH value of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, or 7. In some embodiments, establishment media is maintained at about 22, 23, 24, 25, 26, 27, 28, 29, 30, 31, 32, 33, 34, 35, 36, 37, 38, 39, or 40° C. In some embodiments, stem segments are maintained establishment media at about 8, 9, 10, 11, 12, 13, 14, 15, 16, 17, 18, 19, 20, 21, 22, 23, or 24 hours of light per day. In some embodiments, stem segments are maintained in establishment media at about 20, 30, 40, 50, 60, 70, 80, 90, 100, 150, 200, 250, 300, 350, 400, 450, 500, 550, 600, 650, 700, 750, 800, 850, 900, 950, or 1000 μE of light. In some embodiments, stem segments are maintained in establishment media until shoots appear. In some embodiments, stem segments are maintained establishment media until shoots of about 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm appear. In some embodiments, stem segments are maintained establishment media for about 2 weeks, 3 weeks, 4 weeks, 5 weeks, 6 weeks, 7 weeks, 8 weeks, 9 weeks, or 10 weeks.

### B. Multiplication Media

In the methods described herein, switchgrass shoots grown in establishment media typically are transferred to a multiplication media. In preferred embodiments, multiplication media comprises 1 mg/L CuSO₄.5H₂O, MS plus vitamins, 3% sucrose, 5.0 mg/L BAP, 1.0 mg/L NAA (Naphthaleneacetic acid), 0.25 mg/L TDZ, and 1.0 mg/L PBZ (Paclobutrazol) under conditions comprising 31° C., pH 5.7, 16 hours of light per day, and 35-75 μE of light. In some embodiments, the amount and/or concentrations of hormones may be different as described herein. In some embodiments, the ratio of hormones remains constant. In embodiments where the media is a semi-solid, agar can be used. The agar can be added in about 3, 4, 5, 6, 7, 8, or 9 g/L. In some embodiments, multiplication media comprises MS or MS plus vitamins. In some embodiments, multiplication media comprises about 0.05, 0.01, 0.1, 0.5, 1, 2, 3, 4, 5, 6, 7, 8, 9, or 10 mg/L of CuSO₄.5H₂O. In some embodiments, multiplication media comprises Cu⁺ or Cu⁺⁺ at a μM concentration of about 0.040, 0.100, 0.200, 0.400, 1, 3, 5, 10, 15, 20, 25, 30, 35, 40, 45 μM or ranges therebetween. In some embodiments, CuSO₄.5H₂O can be replaced all or in part with anhydrous CuSO₄, Cu(NO₃)₂, CuCl₂ hydrated or anhydrous, or Cu₂(OH)PO₄. In some embodiments, multiplication media comprises about 0.5%, 1%, 2%, 3%, 4%, 5%, 6%, 7%, 8%, 9%, or 10% sucrose. In some embodiments, multiplication media comprises about 0.25, 0.75, 1.0, 1.5, 2, 2.5, 3, 3.5, 4, or 4.5 mg/L PBZ. In some embodiments, multiplication media comprises about 1, 2, 3, 4, 5, 6, 7, 8, 9, or 10 mg/L BAP. In some embodiments, BAP can be replaced all or in part with kinetin at about 1, 2, 3, 4, 5, 6, 7, 8, 9, or 10 mg/L. In some embodiments, the BAP can be replaced all or in part with zeatin at about 1, 2, 3, 4, 5, 6, 7, 8, 9, or 10 mg/L. In some embodiments, the multiplication media comprises about 1, 2, 3, 4, 5, 6, 7, 8, 9, or 10 mg/L NAA. In some embodiments, NAA can be replaced all or in part with IAA at about 2× the concentration that would be used for NAA, or IBA at about 3×-6× the concentration that would be used for NAA. In some embodiments, NAA can be replaced all or in part with a synthetic auxin such as, but not limited to, 2,4-D or 4-CPU. In some embodiments, multiplication media comprises about 0.01, 0.1, 0.25, 0.75, 1, 1.5, 2, 2.5, 3, 3.5, 4, or 4.5 mg/L TDZ. In some embodiments, multiplication media has a pH of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, or 7. In some embodiments, multiplication media is maintained at about 22, 23, 24, 25, 26, 27, 28, 29, 30, 31, 32, 33, 34, 35, 36, 37, 38, 39, or 40° C. In some embodiments, stem segments are maintained in multiplication media at about 8, 9, 10, 11, 12, 13, 14, 15, 16, 17, 18, 19, 20, 21, 22, 23, or 24 hours of light per day. In some embodiments, stem segments are maintained in multiplication media at about 20, 30, 35, 40, 50, 60, 70, 80, 90, 100, 150, 200, 250, 300, 350, 400, 450, 500, 550, 600, 650, 700, 750, 800, 850, 900, 950, or 1000 μE of light.

### C. Multiplication Rate

In general, if new shoots are broken apart in the multiplication stage multiplication rates increase. In such embodiments, shoots multiply by about 2, 3, or 4 times faster when small shoots are left attached to a large shoot. Using the methods described herein for a 3-week period a multiplication rate of at least 8×, 8.5×, 9×, 9.5×, 10×, 10.5×, 11×, 11.5×, 12×, 12.5× 13×, 13.5×, 14×, 14.5× or 15× is achieved in solid culture. In certain embodiments, a multiplication rate can be maintained for weeks, months or years without significant fluctuation. For liquid culture there is a 50% increase in multiplication rate over solid culture. In general, upland varieties of switchgrass multiply at about 4×, 5×, 6×, or 7× in 2 weeks or 24×, 25×, or 26×, 27×, 28× in 4 weeks. In general, lowland varieties of switchgrass multiply at about 3×, 4×, 5×, or 6× in 2 weeks or 22×, 23×, 24×, 25×, or 26× in 4 weeks.

### D. Rooting Media

Switchgrass shoots from multiplication media can be transferred to a rooting media. In preferred embodiments, rooting media comprises ½ MS plus vitamins, 10% sucrose, 1 mg/L CuSO₄.5H₂O, and 5.0 mg/L NAA under conditions of pH 5.7, 31° C., 16 hours of light per day, and 50-100 μE of light. In some embodiments, the amount and/or concentrations of hormones may be different as described herein. In some embodiments, the ratio of hormones remains constant. In some embodiments, rooting media comprises MS or MS plus vitamins. In some embodiments, rooting media comprises about 0.5%, 1%, 2%, 3%, 4%, 5%, 6%, 7%, 8%, 9%, 10%, 11%, 12%, 13%, 14%, 15%, 16%, 17%, 18%, 19% or 20% sucrose. In some embodiments, rooting media comprises about 0.05, 0.01, 0.1, 0.5, 1, 2, 3, 4, 5, 6, 7, 8, 9, or 10 mg/L CuSO₄.5H₂O. In some embodiments, rooting media comprises Cu⁺ or Cu⁺⁺ at a μM concentration of about 0.040, 0.100, 0.200, 0.400, 1, 3, 5, 10, 15, 20, 25, 30, 35, 40, 45 or ranges therebetween. In some embodiments, rooting media comprises about 0.01, 0.1, 0.25, 0.75, 1, 1.5, 2, 2.5, 3, 3.5, 4, 4.5, 5, 5.5, 6, 6.5, 7, 7.5, 8, 8.5, 9, 9.5, or 10 mg/L NAA. In some embodiments, NAA can be replaced all or in part with IAA at about 2× the concentration that would be used for NAA, or IBA at about 3×-6× the concentration that would be used for NAA. In some embodiments, NAA can be replaced all or in part with a synthetic auxin such as, but not limited to, 2,4-D or 4-CPU. In some embodiments, rooting media has a pH value of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6 or 7. In some embodiments, rooting media is maintained at about 22, 23, 24, 25, 26, 27, 28, 29, 30, 31, 32, 33, 34, 35, 36, 37, 38, 39, or 40° C. In some embodiments, rooting media is maintained for about 4, 5, 6, 7, 8, 9, 10, 11, 12, 13, 14, 15, 16, 17, 18, 19, 20, 21, 22, 23, or 24 hours of light per day. In some embodiments, rooting media is maintained at about 20, 30, 35, 40, 50, 60, 70, 80, 90, 100, 150, 200, 250, 300, 350, 400, 450, 500, 550, 600, 650, 700, 750, 800, 850, 900, 950, or 1000 μE of light.

### III. Propagation of Miscanthus In Vitro

Miscanthus propagation methods described herein generally comprise three main stages: growing cuttings in/on an establishment media, growing shoots in/on a multiplication media, and rooting shoots in/on a rooting media. In some embodiments, the methods do not include a callus phase. In the methods of clonal mass propagation of Miscanthus shoots described herein the starting material is stems segments having at least a portion of a node. The starting material is typically harvested from mature healthy plants and segments of stem are cut (in cross section) that comprise at least one node. Care is taken to sterilize the stems, equipment and growing containers used in the tissue culture methods. In some embodiments, these segments can be cut longitudinally as well. The media described herein and components thereof can be substituted with other media and components that are known in the art. For example, N6 media can be used instead of MS. Any clear and sterile container is suitable for propagation.

### A. Establishment Media

Miscanthus shoots typically are differentiated from a stem section containing at least a portion of a node in an establishment media. In some preferred embodiments, establishment media comprises MS plus vitamins, 30 g/L sucrose (87.7 mM), 3.7 micro-Molar MgCl₂ (750 mg/L of MgCl₂.6H₂O), 5 mg/L BAP (22.2 micro-Molar), and 0.25 mg/L NAA (1.3 micro-Molar) at pH 5.6 with 6 g/L agar for solid cultures, at pH 3.3 for liquid cultures under conditions of 50-100 μE of light, 16 hours of light per day, and 31° C. In some embodiments, the amount and/or concentrations of hormones may be different as described herein. In some embodiments, the ratio of hormones remains constant. In embodiments where the media is a semi-solid, agar can be used. The agar can be added in about 3, 4, 5, 6, 7, 8, or 9 g/L. In some embodiments, establishment media comprises MS, MS plus vitamins, or other media known in the art such as Gamborgs B5 vitamins. In some embodiments, establishment media comprises about 0.5%, 1%, 2%, 3%, 4%, 5%, 6%, 7%, 8%, 9%, or 10% sucrose (where 3% is 30 g/L). In some embodiments, establishment media comprises about 500, 550, 600, 650, 700, 750, 800, 850, 900, or 950 mg/L of MgCl₂.6H₂O.

In some embodiments, establishment media comprises about 1, 2, 3, 4, 5, 6, 7, 8, 9, or 10 mg/L BAP. In some embodiments, BAP can be replaced all or in part with kinetin at about 1, 2, 3, 4, 5, 6, 7, 8, 9, or 10 mg/L. In some embodiments, BAP can be replaced all or in part with zeatin at about 1, 2, 3, 4, 5, 6, 7, 8, 9, or 10 mg/L. In some embodiments, establishment media comprises about 0.1, 0.25, 0.75, 1, 1.5, 2, 2.5, 3, 3.5, 4, or 4.5 mg/L NAA. In some embodiments, NAA can be replaced all or in part with IAA at about 2× the concentration that would be used for NAA, or IBA at about 3×-6× the concentration that would be used for NAA. In some embodiments, NAA can be replaced all or in part with a synthetic auxin such as, but not limited to, 2,4-D or 4-CPU. In some embodiments, establishment media has a pH value of 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6 or 7. In some embodiments, establishment media is maintained at about 22, 23, 24, 25, 26, 27, 28, 29, 30, 31, 32, 33, 34, 35, 36, 37, 38, 39, or 40° C. In some embodiments, stem segments are maintained establishment media at about 8, 9, 10, 11, 12, 13, 14, 15, 16, 17, 18, 19, 20, 21, 22, 23, or 24 hours of light per day. In some embodiments, stem segments are maintained in establishment media at about 20, 30, 40, 50, 60, 70, 80, 90, 100, 150, 200, 250, 300, 350, 400, 450, 500, 550, 600, 650, 700, 750, 800, 850, 900, 950, or 1000 μE of light. In some embodiments, stem segments are maintained in establishment media until shoots appear. In some embodiments, stem segments are maintained establishment media until shoots of about 1 cm, 2 cm, 3 cm, 4 cm, 5 cm, 6 cm, 7 cm, 8 cm, 9 cm, 10 cm, 11 cm, 12 cm, 13 cm, 14 cm, or 15 cm appear. In some embodiments, the stem segments are maintained on establishment media for about 2 weeks, 3 weeks, 4 weeks, 5 weeks, 6 weeks, 7 weeks, 8 weeks, 9 weeks, or 10 weeks.

### B. Multiplication Media

In the methods described herein, the Miscanthus shoots grown in establishment media typically are transferred to a multiplication media. In preferred embodiments, multiplication media comprises MS plus vitamins, 3% sucrose, 3.7 micro-Molar MgCl₂ (750 mg/L of MgCl₂.6H₂O), 5.0 mg/L BAP, 0.25 mg/L TDZ, and 1.0 mg/L PBZ at pH 5.7 with 6 g/L agar for solid cultures, at pH 3.0 for liquid cultures. In some embodiments, the amount and/or concentrations of hormones may be different as described herein. In some embodiments, the ratio of hormones remains constant. In embodiments where media is a semi-solid, agar can be used. The agar can be added in about 3, 4, 5, 6, 7, 8, or 9 g/L. In some embodiments, multiplication media comprises MS or MS plus vitamins.

In some embodiments, multiplication media comprises about 0.5%, 1%, 2%, 3%, 4%, 5%, 6%, 7%, 8%, 9%, or 10% sucrose. In some embodiments, establishment media comprises about 500, 550, 600, 650, 700, 750, 800, 850, 900, or 950 mg/L of MgCl₂.6H₂O. In some embodiments, multiplication media comprises about 0.25, 0.75, 1.0, 1.5, 2, 2.5, 3, 3.5, 4, or 4.5 mg/L PBZ. In some embodiments, multiplication media comprises about 1, 2, 3, 4, 5, 6, 7, 8, 9, or 10 mg/L BAP. In some embodiments, BAP can be replaced all or in part with Kinetin at about 1, 2, 3, 4, 5, 6, 7, 8, 9, or 10 mg/L. In some embodiments, BAP can be replaced all or in part with Zeatin at about 1, 2, 3, 4, 5, 6, 7, 8, 9, or 10 mg/L. In some embodiments, multiplication media comprises about 0.01, 0.1, 0.25, 0.75, 1, 1.5, 2, 2.5, 3, 3.5, 4, or 4.5 mg/L TDZ. In some embodiments, multiplication media has a pH value of 3, 3.5, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6 or 7. In some embodiments, multiplication media is maintained at about 22, 23, 24, 25, 26, 27, 28, 29, 30, 31, 32, 33, 34, 35, 36, 37, 38, 39, or 40° C. In some embodiments, stem segments are maintained in multiplication media for about 0, 1, 2, 3, 4, 5, 6, 7, 8, 9, 10, 11, 12, 13, 14, 15, 16, 17, 18, 19, 20, 21, 22, 23, or 24 hours of light per day. In some embodiments, stem segments are maintained in multiplication media at about 20, 30, 35, 40, 50, 60, 70, 80, 90, 100, 150, 200, 250, 300, 350, 400, 450, 500, 550, 600, 650, 700, 750, 800, 850, 900, 950, or 1000 μE of light.

### C. Multiplication Rate

In general, if new shoots are broken apart in the multiplication stage multiplication rates increase. Using the methods described herein for a 3-week period a multiplication rate of at least 5×, 6×, 7×, 8×, 9×, 10×, 15×, 16×, 17×, 18×, 19×, or 20× if converted to 4 week cycles can be achieved in solid culture. In certain embodiments, a multiplication rate can be maintained for weeks, months or years without significant fluctuation. For liquid culture there is a 50% increase in multiplication rate over solid culture.

### D. Rooting Media

Miscanthus shoots from multiplication media typically are transferred to a rooting media. In preferred embodiments, rooting media comprises ½ MS plus vitamins, 10% sucrose, and 10.0 mg/L NAA at pH 5.7, with 6 g/L agar added for solid cultures. In some embodiments, rooting media comprises MS or MS plus vitamins. In some embodiments, rooting media comprises about 0.5%, 1%, 2%, 3%, 4%, 5%, 6%, 7%, 8%, 9%, 10%, 11%, 12%, 13%, 14%, 15%, 16%, 17%, 18%, 19% or 20% sucrose. In some embodiments, rooting media comprises about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 mg/L NAA. In some embodiments, NAA can be replaced all or in part by IAA at about 2× the concentration that would be used for NAA, or by IBA at about 3×-6× the concentration that would be used for NAA. In some embodiments, NAA can be replaced all or in part with a synthetic auxin such as, but not limited to, 2,4-D or 4-CPU. In some embodiments, rooting media has a pH value of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0. In some embodiments, rooting media is maintained at about 25, 26, 27, 28, 29, 30, 31, 32, 33, 34, 35, 36, 37, 38, 39, or 40° C. In some embodiments, rooting media is maintained at about 0, 1, 2, 3, 4, 5, 6, 7, 8, 9, 10, 11, 12, 13, 14, 15, 16, 17, 18, 19, 20, 21, 22, 23, or 24 hours of light per day. In some embodiments, rooting media is maintained at about 20, 30, 40, 50, 60, 70, 80, 90, 100, 150, 200, 250, 300, 350, 400, 450, 500, 550, 600, 650, 700, 750, 800, 850, 900, 950, or 1000 μE of light.

### IV. Plants Propagated by the Methods of the Invention

In some embodiments, a plant propagated by a method described herein can be a cane species used as an energy crop such as, but not limited to, Panicum virgatum, Miscanthus giganteus, Saccharum sp., Sorghum bicolor, or Arundo donax. For example, a Panicum virgatum plant propagated by a method described herein may be a cultivated variety for feed, forage, fuel, fiber, or ornamental purposes, a Panicum species, a Panicum clone and/or a wild/weedy type of switchgrass. Suitable varieties include, but are not limited to, ‘Alamo’, ‘AM-314’, ‘Blackwell’, ‘BoMaster’, ‘Caddo’, ‘Carthage’, ‘Cave-in-Rock’, ‘Cloud Nine’, ‘Dacotah’, ‘Dakotah’, ‘Dallas Blues’ (U.S. Pat. No. PP11,202), ‘Falcon’, ‘Forestburg’, ‘Grenville’, ‘Haense Herms’, ‘Heavy Metal’, ‘Kanlow’, ‘KY1625’, ‘Miami’, ‘Neb28’, ‘NJ50’, ‘Pangburn’, ‘Pathfinder’, ‘Performer’, ‘PI 414065’, ‘PI 642278’, ‘PMT-785’, ‘Prairie Sky’, ‘REAP 921’, ‘RR1’ (U. S. Pat. No. PP17,944), ‘Rehbraun’, ‘Rotstrahlbusch’, ‘Shawnee’, ‘Shelter’, ‘Sheire’, ‘Shenandoah’, ‘Southlow Michigan’, ‘Squaw, Stuart’, ‘Summer’, ‘Sunburst’, ‘TEM-SLC’, ‘TEM-SEC’, ‘TEM-LoDorm’, ‘Trailblazer’, ‘Wabasso’, ‘Waterfall’ (European Community Plant Variety grant no. 15522), ‘WS4U’, ‘WS8U’, or other known variety such as those listed in the GRIN database. In some embodiments, a switchgrass used in a method described herein is an octaploid, tetraploid, or a hexaploid. In some embodiments, a switchgrass used in a method described herein is an upland variety or a lowland variety. In some embodiments, a switchgrass used in a method described herein is an intra-varietal or inter-varietal hybrid. In some embodiments, a switchgrass used in a method described herein is a plant selected from a population for superior biomass properties. In some embodiments, a switchgrass used in a method described herein is a variety developed from a population through breeding and selection. Such breeding and selection techniques, include, but are not limited to, mass selection, population formation by hybridization using either natural or artificial hybridization, recurrent selection from a base population, mutation breeding, single-seed descent, bulk selection, pedigree selection method, or backcrossing. In some embodiments, a switchgrass used in a method described herein is a variety developed from a population of hybrid origin through selection efforts. In certain embodiments, a switchgrass used in a method described herein is a partially or fully sterile plant. In some embodiments, a switchgrass used in a method described herein is a transgenic plant.

Switchgrass plants clonally propagated as described herein are advantageously used to generate hybrid switchgrass seeds. To generate such seeds, a first clonal population of switchgrass plants and a second clonal population of switchgrass plants are planted in pollinating proximity to each other to allow crossing of the two populations. Switchgrass typically exhibits partial or complete self-incompatibility, and the first and/or the second clonal populations can also exhibit self-incompatibility. In some embodiments, the first clonal population is genetically distinct from the second clonal population and capable of cross pollinating with and fertilizing the first clonal population. Pollination of either the first and/or the second clonal populations can take place by a number of means once flowers have formed. Wind pollination typically is used to facilitate crossing for switchgrass. When each population of plants exhibits self-incompatibility, measures such as male sterility systems or removal of pollen-forming structures on either population are not necessary. Seeds produced on plants of the both the first clonal population and the second clonal population are hybrid switchgrass seeds, and can be harvested separately from each clonal population, or can be harvested together. If harvested separately, the seeds can be subsequently combined if desired.

In some embodiments, one or more clonally propagated varieties can be used as one or more parents in crosses to produce synthetic populations, e.g., a 2 clone synthetic, a 3 clone synthetic, a 4 clone synthetic, a 6 clone synthetic, a 8 clone synthetic, or a 12 clone synthetic. Seeds collected from one or more of the clonally propagated parent plants can be used to plant the first synthetic generation on which selection is applied during the process of development or breeding of a new synthetic variety.

If desired, other means known in the art can be used to allow pollination to occur. Plants of the first population can be pollinated by hand using pollen from plants of the second population and/or plants of the second population can be pollinated by hand using pollen from plants of the first population. As an alternative, pollen-forming structures on plants of the first population are removed manually in order to prevent self-pollination of first plants, thereby permitting manual or natural pollination by pollen from second plants. In yet another alternative, one can use gametocides to inhibit or prevent pollen formation on plants of the first population. As above, seeds produced on plants of the both the first clonal population and the second clonal population may be hybrid switchgrass seeds, and can be harvested separately from each population (and subsequently combined if desired), or can be harvested together. In some embodiments, the hybrid seed harvested from the clonal parents may have a small percentage of self-pollinated seed, for example, 0.50%, 0.25%, 1%, 1.5%, 2%, 3%, 4%, 5%, 6%, 7%, 8%, 9%, or 10%.

A Miscanthus plant propagated by a methods described herein may be a variety, species and/or clone of Miscanthus derived from germplasm that is, for example, wild type, ornamental, fiber, feed, forage, or fuel cultivars, including, but not limited to, Miscanthus × giganteus, Miscanthus sinensis Miscanthus × ogiformis, Miscanthus floridulus, Miscanthus transmorrisonensis, Miscanthus oligstachyus, Miscanthus sacchariflorus, Miscanthus × giganteus ‘Amuri’, Miscanthus x giganteus ‘Nagara’, Miscanthus × giganteus ‘Illinois’, Miscanthus sinensis var. ‘Goliath’, Miscanthus sinensis var. gracillimus, Miscanthus sinensis var. variegates, Miscanthus sinensis var. purpurascens, Miscanthus sinensis var. ‘Malepartus’, Miscanthus sinensis var. ‘Silberfedher’ (aka. Silver Feather), Miscanthus var. ‘Alexander’, Miscanthus var. ‘Adagio’, Miscanthus var. ‘Autumn Light’, Miscanthus var. ‘Cabaret’, Miscanthus var. ‘Condensatus’, Miscanthus var. ‘Cosmopolitan’, Miscanthus var. ‘Dixieland’, Miscanthus var. ‘Gilded Tower’ (U.S. Pat. No. PP14,743), Miscanthus var. ‘Gold Bar’ (U.S. Pat. No. PP15,193), Miscanthus var. ‘Gracillimus’, Miscanthus var. ‘Graziella’Miscanthus var. ‘Grosse Fontaine’, Miscanthus var. ‘Hinjo aka Little Nicky’™, Miscanthus var. ‘Juli’, Miscanthus var. ‘Kaskade’, Miscanthus var. ‘Kirk Alexander’, Miscanthus var. ‘Kleine Fontaine’, Miscanthus var. ‘Kleine Silberspinne’ (aka. ‘Little Silver Spider’), Miscanthus var. ‘Little Kitten’, Miscanthus var. ‘Little Zebra’ (U.S. Pat. No. PP13,008), Miscanthus var. ‘Lottum’, Miscanthus var. ‘Malepartus’, Miscanthus var. ‘Morning Light’, Miscanthus var. ‘Mysterious Maiden’ (U.S. Pat. No. PP16,176), Miscanthus var. ‘Nippon’, Miscanthus var. ‘November Sunset’, Miscanthus var. ‘Parachute’, Miscanthus var. ‘Positano’, Miscanthus var. ‘Puenktchen’ (aka ‘Little Dot’), Miscanthus var. ‘Rigoletto’, Miscanthus var. ‘Sarabande’, Miscanthus var. ‘Silberpfeil’ (aka. Silver Arrow), Miscanthus var. ‘Silverstripe’, Miscanthus var. ‘Super Stripe’ (U.S. Pat. No. PP18,161), Miscanthus var. ‘Strictus’, or Miscanthus var. ‘Zebrinus’.

In certain embodiments, a plant propagated by the methods described herein can be a hybrid of different species, varieties of a specific species, or clones of a variety (e.g., Saccharum sp. × Miscanthus sp., Saccharum sp. × Sorghum sp., or Panicum virgatum v. Alamo × Panicum virgatum v. Summer).

### IV. Advantages

A drawback of the use of known propagation conditions is a limited shoot multiplication rate. Optimization of media and culture conditions does not necessarily result in commercially acceptable multiplication rates. Even in combination, the previously adopted techniques have failed to increase multiplication rates. Alexandrova described how to multiply up to 500 shoots from one plant in 8 weeks (12 weeks to plant) at 29° C. and used only one plant hormone Benzylaminopurine (BAP) for multiplication of switchgrass shoots (Alexandrova et al. 1996). However, it has been discovered that 31° C. is a preferable temperature for multiplication of switchgrass shoots. It has also been discovered that liquid medium is preferable for increased multiplication. Applicants optimized multiplication of Switchgrass shoots with a combination of several plant hormones Benzylaminopurine+Naphthaleneacetic acid+Thidiazuron+Paclobutrazol (BAP+NAA+TDZ+PBZ). When a presumed optimal mixture of hormones, temperature and liquid medium conditions were used, it was unexpectedly found that there was a problem of low multiplication rates in liquid culture instead of the expected additive effect of increased multiplication rates (2.7×-3.9× every three weeks instead of an expected additive effect of 6× every 3 weeks). The combined optimized temperature and hormone mixture in liquid culture conditions resulted in shoots longer than 1 cm dying, decreasing the shoot multiplication rate.

It was discovered that an increase in the amount of 6 micro-elements restored growth to shoots and prevented shoot death and, in particular, an increase in the amount of CuSO₄.7H₂O added to the media increased multiplication rates increased up to about 7× every 3 weeks. MS media contains 0.025 mg/L CuSO₄.7H₂O. An increase of up to about 10× every 3 weeks in multiplication rate was achieved when shoots were broken apart every 2-3 weeks.

Using methods described herein, it is possible to achieve an increase in multiplication over published methods. For example, 1 million plants can be propagated from a single plant in about 24 weeks (1 million shoots in about 16 weeks) and/or a sustained continual propagation rate of about 10× every 3 weeks. The disclosed methods can increase the shoot multiplication rate of switchgrass in culture media. The disclosed methods can also improve the efficiency of clonal propagation of switchgrass, by ensuring that a large majority of the in vitro propagated plants remain viable, and increases the multiplication rate. This increased level of clonal propagation is useful for commercial applications such as seed production. Especially for an obligate outcross species like switchgrass, with few elite lines or cultivars available, clonal populations desirable as parents of hybrid seeds need to be multiplied quite rapidly if commercial levels of seed production are to be accomplished within acceptable timelines.

In propagating Miscanthus stem nodes in culture media, Nielsen used high amounts of Thidiazuron (TDZ) (1.65 mg/L), which is a cost limiting hormone additive in shoot multiplication growth media (Nielsen et. al. 1993; Nielsen et. al. 1995). In contrast it has been discovered that with such high levels of TDZ, multiplied shoots were thinner and shoot production was less in comparison to shoot multiplication in media having amounts of TDZ of about 0.25 mg/L TDZ where media was maintained at 31° C.

The invention will be further described in the following examples, which do not limit the scope of the invention described in the claims.

### VI. Examples

### Example 1

### In Vitro Clonal Propagation of Switchgrass

To establish switchgrass shoots into sterile culture for propagation, several varieties of switchgrass were used, including PI 642278 and Alamo. Healthy greenhouse grown plants were collected. A 20% Clorox® solution with Silwet was made (in a 1 L bottle add 200 ml Clorox® bleach to 800 ml nano-pure H₂O then add 1 ml Silwet). A solution of 70% ethanol was sprayed on pruning shears and allowed to dry to sterilize the cutting instruments. Switchgrass cane and/or tiller sections having a node were cut approximately 1 cm below the node and 1-2 cm above the node. Harvested nodes were placed in an autoclaved 500 ml bottle.

To sterilize the cuttings, 300-400 ml 70% ethanol was added to cut nodes for 1 minute. Ethanol was poured off and 300-400 ml of 20% Clorox® solution was added to the bottle with the nodes and placed on a shaker for one hour. After 1 hour, the bottle was taken under a laminar-flow hood and cleaned off with 70% ethanol. The Clorox® was poured off into a waste beaker. 300-400 ml of sterile water was added to the bottle and mixed for 1 minute. The water was poured off (using a fresh sterile Petri plate to hold the nodes in the bottle) and refilled with more water. The bottle contents were mixed for 5 minutes. This step of rinsing was repeated twice. After the water has been poured off the 3rd time, the nodes were placed into a sterile Petri plate using sterile forceps. The cuttings with nodes were then cut length-wise. A node was held with sterile forceps and the node was cut from the top with the sterile scalpel.

For the establishment phase, media was prepared containing MS plus vitamins (Phytotechnologies™ Product ID-M519), 3% sucrose, 1 mg/L CuSO₄.5H₂O, and 6 g/L Agar at pH 5.7. The media was autoclaved and filter-sterilized hormones 5 mg/L 6-Benzylaminopurine (BAP) (Phytotechnologies™ Product ID-B800) and 0.25 mg/L Thidiazuron (TDZ) (Phytotechnologies™ Product ID-T888) were added under the laminar-flow hood (values represent final concentrations in media, not stock concentrations). The resulting mixture was poured into 100×20 mm Petri plates, 35-40 ml each (1 L=approximately 26 plates). Both halves of the split node were placed onto the described establishment media for switchgrass. Each was placed with the cut side against the media surface. Up to 5 pairs were placed into one Petri plate and spread out as much as possible. Each Petri plate was then covered and wrapped with Parafilm or sealing film, labeled, and placed into a growth chamber set to 31° C., 16 h day, with 50-100 μE of light. For one month, plates were checked for contamination every day and where contamination was observed clean-looking shoots were removed to fresh media. Two weeks after sterilization, not all nodes contained a growing bud. If the new shoot was greater than 3 cm long, it was removed from the node and cut down to about 1 cm and placed onto fresh media. If the shoot was less than 3 cm, the node/shoot was transferred together to fresh media. Using 2 forceps, or 1 forceps and 1 scalpel, the old leaf sheath covering the base of the new shoot was pealed back. The shoot was carefully cut off the node as close to the node as possible placed into an empty sterile Petri plate and cut about 1 cm above the base. The shoot was placed into fresh media and the plate was wrapped and labeled. Two to three weeks after shoots were cut away from the node they were transfer to a shoot multiplication media for switchgrass.

Plant cuttings were propagated in both semi-solid and liquid media for shoot multiplication, the example presented herein relates to both solid culture in Petri plates and liquid culture in flasks having vented caps with a 0.2 micrometer filter or containers unless otherwise indicated. The switchgrass shoot multiplication media (solid cultures) consisted of MS plus vitamins (full strength), 3% sucrose, and 1 mg/L CuSO₄.5H₂O at pH 5.7 with 6 g/L agar. After autoclaving, the filter-sterilized hormones 5.0 mg/L BAP, 1.0 mg/L Naphthaleneacetic acid (NAA) (Phytotechnologies™ Product ID-N600), 0.25 mg/L TDZ, and 1.0 mg/L Paclobutrazol (PBZ, a gibberellic acid inhibitor) (Phytotechnologies™ Product ID-P687) were added under a laminar-flow hood and poured into 100×20 mm Petri plates, 35-40 ml each (1 L=approximately 26 plates). The switchgrass shoot multiplication media (liquid cultures) consisted of MS+vitamins (full strength), 3% sucrose, and 1 mg/L CuSO₄.5H₂O at pH 5.7. After autoclaving, the following filter-sterilized hormones 5.0 mg/L BAP,1.0 mg/L NAA, 0.25 mg/L TDZ, and 1.0 mg/L PBZ were added under a laminar-flow hood and poured into 100×20 mm Petri plates, 35-40 ml each (1 L=approximately 26 plates).

For semi-solid culture shoot multiplication, culture vessels were thoroughly sprayed with 70% ethanol and dried under the sterile hood before opening them. Healthy shoots were taken from establishment media and placed one clump at a time into sterile Petri plates. Sterile forceps and scalpel were used to separate clumps of shoots in to smaller clumps. Once clumps were separated, any shoots that were longer than 3 cm were cut down to 2-3 cm long and placed into fresh media. Up to 10 small clumps (a small clump has no more than 5-7 shoots) of shoots were placed into each 100×20 Petri plate with media. Plates were sealed, labeled and placed into a growth chamber at 31° C., 16 hr day, 35-75 μE of light. Shoots were transferred (and sub-cultured if necessary) to fresh media every 2 weeks. Multiplication rates were maintained continuously for 8 months at which point the experiment was intentionally ended, not because of any decrease in multiplication or other problems with shoots.

For liquid culture shoot multiplication, all flasks or growth containers used were sprayed with ethanol and let dry before opening. Healthy shoots were taken from establishment media and separated as described above except shoots were cut to 4-5 cm long. Up to 25 small shoot clumps (100 shoots) were placed into each flask or container. 20-50 ml liquid media was added to each flask. The flask or container was then sealed (excluding the vented cap) and placed into a growth chamber at 31° C., 16 hr day, 35-75 μE of light. Shoots were transferred (and sub-cultured if necessary) to fresh media every 2 weeks.

Rooting of multiplied shoots in solid culture was achieved using the following protocol. Culture vessels were sterilized with 70% ethanol and let dry under a sterile hood before opening them. Shoots greater than 3 cm tall were transferred to rooting media and smaller shoots were first transferred to pre-rooting media for 2-3 weeks. The switchgrass pre-rooting media (solid or liquid cultures) consisted of MS plus vitamins (full strength), 3% sucrose, and 1 mg/L CuSO₄.5H₂O at pH 5.7 with 6 g/L agar for solid media. After autoclaving, the filter-sterilized hormone 2.0 mg/L BAP was added under a laminar-flow hood. For semi-solid cultures, the media mixture was poured into 100×20 mm Petri plates, 35-40 ml each (1 L=approximately 26 plates). The switchgrass rooting media consisted of ½ MS plus vitamins, 10% sucrose, and 1 mg/L CuSO₄.5H₂O at pH 5.7 with the addition of 6 g/L agar for solid cultures. After autoclaving, the filter-sterilized hormone 5.0 mg/L NAA was added under a laminar-flow hood and poured into 100×20 mm Petri plates, 35-40 ml each (1 L=approximately 26 plates).

Shoots were either left as clumps for rooting, or shoots separated for individual rooting. Shoots left in clumps during rooting can be separated into smaller clumps (or singular shoots) when they are being moved into soil. Most of the larger shoots had several roots after 2 weeks. After 2 weeks, any shoots without roots were transfer to fresh rooting media for another 2 weeks. Shoots with roots were moved into soil. To help keep down fungal growth, excess plant media was washed off from the roots with sterile water before putting shoots into soil. Plants were covered with a clear cover and placed in a greenhouse at about 30° C. After 7-10 days, plants were uncovered. Plants were about 30 cm tall in 4 weeks.

For liquid culture root establishment, the protocol involved changing the media once a week, except that the media was replaced with pre-rooting media for 2 weeks and then replaced with rooting media for another 2 weeks.

### Results

Table 1 shows the 3-week multiplication factors for switchgrass shoots grown in vitro for an upland octaploid switchgrass (PI 645578) and a lowland tetraploid switchgrass (Alamo). Shoots were grown in liquid medium at 31° C. with the following hormones (mg/L): 5.0 BAP, 1 NAA, 0.25 TDZ & 1 PBZ.

TABLE 1
In vitro Multiplication Rates for Two Varieties of
Switchgrass

| Alamo | PI 645578 (North Dakota) |
|---|---|
| 10.9x | 11.8x |

### Example 2

### In Vitro Clonal Propagation of Miscanthus

Stems of actively growing Miscanthus × giganteus having dormant nodes were harvested and sterilized using 1.5% sodium hypochlorite (25% bleach) solution followed by three rinses using sterile water. Stem segments comprising nodes were cut to 3 cm long and then cut longitudinally down the center of the each stem. The prepared stem portions were then placed under growth conditions of 50 μE of light and a 16 h day. The temperature was 31° C. Stem segments were grown in 100 m×20 mm Petri plates for solid culture, however any suitable sterile container can be used for solid or liquid culture. Time between sub-culturing was 14-21 days. Shoots were cut to 3-4 cm in length when sub-culturing.

The media used for the above-described growth conditions comprised MS medium plus vitamins (full strength) (Phytotechnologies™ Product ID-M519), 30 g/L sucrose (87.7 mM), 3.7 micro-Molar MgCl2 (750 mg/L of MgCl₂.6H₂O), pH 5.6 for semi-solid media, pH 3.3, and 6 g/L agar. The following hormones were added after autoclaving of the media. During the establishment phase (first 8 weeks), hormones added to the establishment media included 5 mg/L BAP (22.2 micro-Molar) and 0.25 mg/L NAA (1.3 micro-Molar). Cultures were checked daily and clean shoots were removed from containers having contamination and placed into fresh media. Two weeks after sterilization, germinating nodes were checked. New shoots greater than 4 cm long were removed from the node, cut to about 2 cm and placed onto fresh media. If the shoot was less than 3 cm the node/shoot was transferred together to fresh media. Two to three weeks after shoots were cut away from the node they were transferred to fresh media.

Following the establishment phase plant material was placed in either a liquid or solid multiplication media having the following components: MS plus vitamins, 3% sucrose, 750 mg/L MgCl₂.6H₂O, 5 mg/L BAP (22.2 micro-Molar), 0.25 mg/L TDZ (1.14 micro-Molar), 1 mg/L Paclobutrazol (PBZ) (3.4 micro-Molar), 6 g/L agar (for solid cultures), pH 5.6 for solid cultures and pH 3.3 for liquid cultures. Solid culture shoots were transferred every 2 weeks, but liquid media needed transferring every week (subculture every 2-4 weeks). Shoot clumps were separated once shoots grew to about 4 cm in length and any shoots that were longer than 4 cm were cut to 3 cm long and placed into fresh media.

Following the multiplication phase plant material was placed on a ½ MS plus vitamins rooting media. Liquid or semi-solid media was used having 10% sucrose, gelling agent, and MgCl₂ as in multiplication media, with the additions of 10 mg/L NAA (53.7 micro-Molar), a pH of 5.6 (when semi-solid media was used), and a pH of 6.5 (when liquid culture was used) with 2 g/L MES buffer added.

### Results

Using the media and steps described above, multiplication rates of 8-9× every 3 weeks or 17-18× if converted to 4 week cycles were achieved.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of multiplying switchgrass shoots, the method comprising growing a plurality of switchgrass shoots at about 31° C. in a multiplication media comprising Cu⁺⁺ at a concentration of from about 0.040 μM to about 45 μM.

2. The method of claim 1, wherein said Cu⁺⁺ concentration is 0.040, 0.1, 0.20, 0.40, 1, 3, 5, 10, 15, 20, 25, 30, 35, 40, or 45 μM.

3. The method of claim 1, wherein the switchgrass shoots multiply during the growing step at a rate of at least ten-fold in three weeks.

4. The method of claim 3, wherein the switchgrass shoot multiplication rate is continuous.

5. The method of claim 1, wherein the Cu⁺⁺ is added to the media as CuSO₄.7H₂O.

6. The method of claim 1, wherein the multiplication media further comprises MS plus vitamins, 3% sucrose, hormones BAP, TDZ, NAA, and PBZ in a concentration ratio of 20:1:4:4.

7. The method of claim 6, wherein the plurality of shoots are grown under conditions of pH 5.7, 16 hours of light per day, and 35-75 μE of light.

8. The method of claim 1, wherein said plurality of shoots are formed by differentiation from a stem section containing at least a portion of a node in an establishment media, prior to said growing on multiplication media.

9. The method of claim 8, wherein the establishment media comprises MS plus vitamins, 3% sucrose, 1 mg/L CuSO₄.5H₂O, 5 mg/L BAP and 0.25 mg/L TDZ.

10. The method of claim 9, wherein the establishment media further comprises 1.0 mg/L NAA and 1.0 mg/L PBZ.

11. The method of claim 9, wherein the establishment media further comprises 6 g/L agar.

12. The method of claim 8, wherein said plurality of shoots are grown under conditions of pH 5.7, 16 hours of light per day, and 50-100 μE of light.

13. The method of claim 8, wherein the shoots are removed from the establishment media after two weeks and separated from the node.

14. The method of claim 8, wherein shoots are removed from the establishment media at 3 cm long and separated from the node.

15. The method of claim 1, wherein the plurality of shoots are grown in a rooting media after the multiplication step, the rooting media comprising MS plus vitamins, 3% sucrose, 1 mg/L CuSO₄.5H₂O, and 1.0 mg/L NAA, and the plurality of shoots are grown under conditions of pH 5.7, 16 hours of light per day, and 50-100 μE of light.

16. The method of claim 15, wherein the rooting media further comprises 6 g/L agar.

17. The method of claim 15, further comprising the step of regenerating a plurality of plants from the shoots placed on the rooting media, wherein the shoots placed on the rooting media are multiplied in the multiplication step from a single stem section containing at least a portion of a node.

18. A plant made by the method of claim 17, or a progeny thereof.

19. A method of making hybrid switchgrass seeds, the method comprising:
a) planting a first clonal population of switchgrass plants, the plants regenerated from switchgrass shoots according to the method of claim 14;
b) planting a second clonal population of switchgrass plants in pollinating proximity to the first clonal population, the second clonal population of plants regenerated from switchgrass shoots according to the method of claim 14; and
c) collecting seed produced on the first clonal population, the second clonal population, or both the first and second clonal populations.

20. The method of claim 19, wherein the switchgrass plants of the first clonal population are male sterile plants and seed is collected from plants of the first clonal population.

21. The method of claim 19, wherein the switchgrass plants of the first clonal population are transgenic plants.

22. A method for clonally propagating Miscanthus under conditions of 50-100 μE of light, 16 hours of light per day and 31° C., comprising:
a) growing stem sections comprising at least a portion of a node in an semi-solid establishment media at pH 5.6 with 6 g/L agar or a liquid establishment media at pH 3.3 to form shoots, said establishment media comprising MS plus vitamins, 30 g/L sucrose (87.7 mM), 3.7 micro-Molar MgCl₂ (750 mg/L of MgCl₂.6H₂O), 5 mg/L BAP (22.2 micro-Molar), and 0.25 mg/L NAA (1.3 micro-Molar);
b) growing shoots formed in step a) in a semi-solid multiplication media at pH 5.7 with 6 g/L agar or a liquid multiplication media at pH 3.0 to multiply said shoots, said multiplication media comprising MS plus vitamins, 3% sucrose, 5.0 mg/L BAP, 0.25 mg/L TDZ, and 1.0 mg/L PBZ; and
c) growing shoots formed in step b) in a rooting media comprising MS plus vitamins, 3% sucrose, and 10.0 mg/L NAA with 6 g/L agar at pH 5.7 to form Miscanthus plantlets.

23. The method of claim 22, wherein the shoots in step b) multiply at a rate of at least eight-fold every three weeks.

24. The method of claim 22, wherein the shoots in step b) multiply at a rate that is continuous.

25. The method of claim 22, wherein the stem sections are 3 cm long.

26. The method of claim 22, wherein the stem sections are cut in cross-section and longitudinally.

27. A plant made by the method of claim 22 or a progeny thereof.
